(19) United States
(12) Patent Application Publication
(10) Pub. No.: US 2024/0094185 A1
(43) Pub. Date: Mar. 21, 2024

(54) CARBON MONITORING PROCESS FOR PYROLYSIS REACTIONS

(71) Applicant: Carba Inc., Edina, MN (US)

(72) Inventor: Andrew JONES, Edina, MN (US)

(73) Assignee: Carba Inc., Edina, MN (US)

(21) Appl. No.: 18/468,269

(22) Filed: Sep. 15, 2023

Related U.S. Application Data
(60) Provisional application No. 63/407,194, filed on Sep. 16, 2022.
(60) Provisional application No. 63/407,197, filed on Sep. 16, 2022.

(51) Int. Cl. G01N 33/24; G01N 31/12

(52) U.S. Cl. CPC G01N 33/241; G01N 31/12

(57) ABSTRACT

A process is hereby provided for monitoring the progress of pyrolysis reactions. Effluent from the pyrolysis reaction is fed to a catalytic reactor to catalytically combust the carbon in the pyrolysis exhaust gases to carbon dioxide. The CO₂ is then subsequently measured. This allows for the efficient and accurate quantification of total organic carbon evolved from the reaction with time. This provides a more accurate surrogate for reaction progress than time or temperature. This technique is more accurate and provides faster response than other analytical techniques such as mass spectrometry and flame ionization detection. More accurate reaction monitoring allows for improved reaction control, optimization and the ability to utilize variable feedstocks.

### CROSS-REFERENCE TO RELATED APPLICATIONS

This application claims priority to U.S. Provisional Application Ser. No. 63/407,194 filed Sep. 16, 2022, and U.S. Provisional Application Ser. No. 63/407,197, filed Sep. 16, 2022, the complete disclosures of which are incorporated herein by reference in their entireties.

### FIELD

The present application relates to the reduction of carbon dioxide in the atmosphere and a reaction monitoring process for the pyrolysis equipment. The present process provides a more accurate and cost-effective means of monitoring reaction progress.

### BACKGROUND

The continued emissions of gasses into Earth's atmosphere has emerged as an existential crisis in the 21st century. Over a century of combustion of fossil fuels has accumulated excess carbon dioxide raising the composition to 414 ppm in 2021, far above the ˜300 ppm CO₂ composition at the beginning of the 20th century. At the current rate of carbon accumulation in the atmosphere, carbon dioxide concentrations in the atmosphere could produce a 5° C. warming by the end of the century, excluding any mitigation. The resulting climate change has been predicted for a century, with increasing sea levels, more powerful storms, and more intense and frequent droughts and heat waves. Catastrophe is predicted for wildlife, the environment, and human civilization, with increased extinction of species, human migration away from climate-impacted areas, and substantial economic costs imposed by these global changes.

Climate change will have sweeping effects on human society including the economy and financial sector. Climate-related shifts in the physical environment can slow economic growth and increase the likelihood of disruption and reductions in output, employment, and business profitability. Humans and animals will face new survival challenges because of climate change. Storms, heat waves, more frequent and intensive droughts, melting glaciers, rising sea levels and warming oceans can directly harm animals, destroy places they live, and wreak havoc on people's livelihoods and communities. Carbon dioxide in the atmosphere has a major impact in creating such climate change.

Strategies to address climate change have included new policies to limit carbon emissions, changes in human behavior to low-carbon activities, and new technologies to adjust the relationship of civilization with carbon waste emissions. Electricity generated from wind turbines and solar photovoltaics provide lower-carbon alternatives to conventional steam-generator systems powered by fossil fuel combustion. For power utilization, hydrogen- or battery-powered vehicles can operate from electrical power sources without direct CO₂ emissions, while heat pumps, automation, and high-efficiency lighting reduce the environmental impact of buildings. While these technologies provide solutions to mitigate future carbon emissions, the urgent threat of the climate change problem also requires addressing historical CO₂ accumulation in the atmosphere via carbon removal.

Removing over a century of carbon emissions from the atmosphere is challenged by the scale of carbon mass that must be reclaimed. This challenge is exacerbated with the ongoing increase in emissions, with global CO₂ emissions of 33 Gt-CO₂ in 2020 alone (˜9 gigatonnes carbon). Human civilization has already emitted several hundred petagrams (100 pgC is 100·10¹⁵ gC or 100 gigatonnes) of carbon since the 19th century, with forecasted cumulative emissions of an exagram (1000·10¹⁵ gC, 1000 gigatonnes) of carbon by 2050. For perspective, a gigatonne of graphite (density 2260 kg m⁻³) would fill about 180,000 Olympic swimming pools (almost half of a cubic kilometer). For the total amount of carbon anticipated by 2050 at the current rate of emission, about a thousand gigatonnes, the volumetric space approximately the equivalent of Lake Erie (483 km³) is required to store carbon in the form of dense graphite. Achieving this volume of CO₂ collection, processing, and storage in less than half a century will require extraordinary effort using technology that can be rapidly implemented at low cost and a massive scale.

Carbon management technologies must be able to capture carbon dioxide from the atmosphere and store it with a level of permanence relevant to: (i) the time scale of climate change, (ii) the quantity of carbon to be removed, and (iii) the economics per tonne of captured carbon. These processes remain in development but can be classified by their general approach to obtaining carbon, the chemical steps for converting it to some final form, and the type of carbonaceous product and associated degree of permanence. The complexity of the carbon accumulation and storage technologies has led to an expansive array of process concepts, and an even more complex combination of words and phrases to classify the process classes.

One common process method is “direct air capture” (DAC), whereby carbon dioxide is removed from the air via a human-manufactured accumulation process. Carbon dioxide exists within the atmosphere at several hundred parts per million, and the first step requires the collection of carbon dioxide molecules together. Carbon dioxide is dispersed among thousands of oxygen and nitrogen molecules, extending up miles into the atmosphere. This carbon dioxide can be accumulated on surfaces in a process called ‘adsorption’, with engineered surfaces designed for selective chemisorption and subsequent desorption. Alternatively, carbon dioxide can be accumulated in liquids or solids by ‘absorption’, after which the resulting enriched solid or liquid is stored, or the CO₂ is recovered. In a third option, CO₂ is directly separated from air by methods such as membranes. All of these classes of CO₂ capture require substantial energy to trap, accumulate, and release enriched CO₂ from the atmosphere; this ‘entropy penalty’ constitutes substantial operating and capital costs associated with the CO₂-accumulating equipment.

The existing methods of removing CO₂ from the atmosphere have the commonality of accumulating or concentrating gases from the atmosphere. At ˜400 ppm CO₂ concentration in the atmosphere, the theoretical energy requirement to accumulate to a highly concentrated stream of 99 mol % CO₂ is about 20 kJ mole-CO₂⁻¹. But real DAC technologies are inefficient, with a realistic 5% efficiency CO₂ capturing technology requiring ˜400 kJ mole-CO₂⁻¹. This energy requirement is equal to ˜2500 kWhr Tonne-CO₂⁻¹, or the equivalent energy generated by a large 3 MW wind turbine operating for one hour. Using more conventional power sources, this exceeds the 158 kJ of electrical energy generated per mole of CO₂ produced by coal generation or is close to parity with natural gas fired electricity generation of 396 kJ mole-CO₂⁻¹. All of these comparisons indicate that a substantial amount of energy is required to drive DAC technologies, and that renewable energy would have a larger impact on CO₂ reduction when used to replace coal power plants rather than power DAC.

The individual direct-air capture technologies exhibit power requirements comparable to the theoretical requirement adjusted for 5% efficiency. The use of sorbents for direct air capture has been estimated as high as 12 GJ Tonne-CO₂⁻¹ equivalent, corresponding to ˜3300 kWhr Tonne-CO₂⁻¹. By this method, air blown over high surface area materials has CO₂ removed by adsorption to the surface; in a second phase, the high surface area materials undergo new conditions such as higher temperature to promote desorption as a high concentration CO₂ effluent stream. These methods include additional processing costs such as regeneration of the binding site, compression or purification of the effluent CO₂-rich stream, and/or pre-treating of the air input stream such as compression to enhance the rate or extent of adsorption. The conditions of operation of adsorption systems are dictated by the material adsorbents, with engineered binding sites consisting of many structures including Lewis acids incorporated into high surface area materials such as resins, zeolites, carbons, or metal organic frameworks. These newer materials combined with improved process designs can lower the energy requirements to ˜1600 kWhr Tonne-CO₂⁻¹ (FIG. 2-(b)), albeit with lower concentrations of CO₂ in the effluent stream (70-80%) that lead to higher compression and injection costs, and higher material costs.

Alternatively, direct air capture has assessed absorption technologies that accumulate carbon dioxide in a liquid, solid, or multi-phase fluid system; this approach also requires substantial energy input consistent with low overall efficiency. Similar to adsorption technologies, absorption methods expose liquids or solids to air leading to uptake and concentration of carbon dioxide, after which the solid or liquid sorbent is moved to a new vessel with conditions promoting the release and concentration of CO₂. Sorption processes are designed with respect to the characteristics of CO₂ in the sorbent material, with engineered fluids achieving improved control of carbon dioxide capture and release. For example, a continuous process utilizing an aqueous KOH sorbent captures and concentrates CO₂ with an energy input of ˜2440 kWhr Tonne-CO₂⁻¹. Similarly, an amine-based continuous process was modeled to consume ˜2990 kWhr Tonne-CO₂⁻¹, despite several decades in the development of amine scrubbing technology.

As a third option, carbon dioxide separation via membranes aims to concentrate carbon dioxide in the air to a CO₂-rich permeate stream. These membranes are designed for CO₂ solubility, often containing amines, with a selectivity target of CO₂/N₂ of ˜100-200. These processes require substantial unit operation integration, providing compressed gases in systems that are sometimes multiple stages. In one example, commercial membranes separating CO₂ from air use as much energy as 18,000 kWhr Tonne-CO₂⁻¹. Alternatively, DAC processes using a more advanced membrane can achieve comparable CO₂ separation with much less energy (˜3,000 kWhr Tonne-CO₂⁻¹). Despite these improvements, CO₂ capture from air using membranes remains an energy-intensive methodology with several challenges.

Membranes, adsorbents, and absorbents all require substantial energy input to acquire, separate, and concentrate CO₂ from the air. Moreover, this energy input only advances the carbon capture sequence to a concentrated stream of CO₂; energy contributions to convert or place this gaseous carbon in a permanent location remain substantial. One commonly proposed end-point of CO₂ is compression and injection into existing geological storage sites. Alternatively, carbon dioxide can be reacted to a variety of products including formation of carbonates with calcium oxides, reduction to methanol for chemical and polymer applications, or in direct use applications such as foods or enhanced oil recovery. In all cases, an energy penalty of several hundred kilojoules must be provided to convert each mole of CO₂. Independent of the final CO₂ conversion process, all of the DAC methods are significantly limited energetically and economically by the upfront entropy penalty of accumulating and concentrating carbon dioxide.

Individuals and companies can work to reduce their carbon emissions; however, permanent removal of carbon dioxide from the atmosphere and the environment is the only way to limit the worst effects of climate change and get back to pre-industrial CO₂ levels. A better solution for such removal of CO₂ is needed to aid in reducing the sweeping shifts of climate change. While pyrolysis of biomass is a promising approach to storing carbon long term, a better method to monitor the progress of reactions with different feedstocks, other than just temperature and time, is needed to realize the promise.

### SUMMARY

A process is hereby provided for monitoring the progress of pyrolysis reactions that are critical for efficient gigatonne removal of carbon dioxide from the atmosphere. Effluent from the pyrolysis reaction is fed to a catalytic reactor to catalytically combust the carbon in the pyrolysis exhaust gases to carbon dioxide. The CO₂ is then subsequently measured. This allows for the efficient and accurate quantification of total organic carbon evolved from the reaction with time. This provides a more accurate surrogate for reaction progress than time or temperature. More accurate reaction monitoring allows for improved reaction control, optimization and the ability to utilize variable feedstocks.

In another embodiment, provided is a reactor for converting biomass to a solid carbon product. The reactor comprising a reactor chamber that can be heated to pyrolysis temperatures with a twin screw conveyer in the reactor chamber. A catalytic reactor is in connection with the reactor chamber such that the catalytic reactor receives effluent from the reactor. A CO₂ detector is in connection with the catalytic reactor such that the CO₂ detector receives effluent from the catalytic reactor. The measurement of the CO₂ helps to monitor the progress of the reaction in the reactor.

### BRIEF DESCRIPTION OF THE FIGURES OF THE DRAWINGS

FIG. 1 depicts the measurement of CO₂ as a function of time for a reaction run at 400° C.

FIG. 2 depicts a reactor of one embodiment of the present application.

FIG. 3 schematically depicts the screw conveyers of the reactor.

FIG. 4 schematically depicts one embodiment of the screw conveyers from an end view to better show preferred cut and folded flights of the screw conveyers.

FIG. 5 schematically depicts, in one embodiment, the containment around the reactor for heat transfer fluid such as molten salt heat transfer fluid.

### DETAILED DESCRIPTION

The present process converts biomass waste, e.g., tree and plant waste, to a solid carbon product. A reactor is used to pyrolyze the biomass. The tree and plant waste can comprise natural products obtained from trees and plants, including woodchips, corn stover, nut shells, seaweed, agricultural debris, etc. Thus, various different biomass waste will be used in the conversion, with the reaction conversion often taking different lengths of time based on the feed. Efficiency is achieved by the present process of monitoring any such conversion reaction, regardless of reactor, regardless of feed, so that understanding the character of the reaction and when it is complete allows one to adjust or more importantly shut down the reaction when a desired product is achieved.

A method of monitoring the reaction progress is provided in the present process. These reactors are used with a variety of feedstocks in a variety of environments with varying moisture content and chemistries. So, there is a need to monitor reaction progress for each batch to determine when to stop. To do this a novel measurement technique has been designed that is effective and inexpensive.

A fraction of the effluent of the reactor is sent to a small catalytic reactor containing a combustion catalyst (e.g. Pt, Pd, Rh, and/or Co), the reactor is ideally heated to 450-650° C., and the effluent of this catalytic reactor is sent to a CO₂ detector, e.g., an NDIR CO₂ detector (e.g. https://www.co2 meter.com/products/cozir-2000-ppm-co2-sensor?variant=209887967). In general, the amount of effluent passed to the catalytic reactor ranges from 1-5 sccm. Clean air or oxygen (scrubbed of CO₂) is fed to the reactor to provide oxygen needed for combustion. This allows one to easily monitor the total organic carbon content coming off the reactor, which provides information about the reaction progress. All organic molecules that reach the reactor are combusted to CO₂. From this measurement, temperature probes, and time, sufficient information is provided to monitor the reaction progress and determine when to end the reaction for a given feedstock.

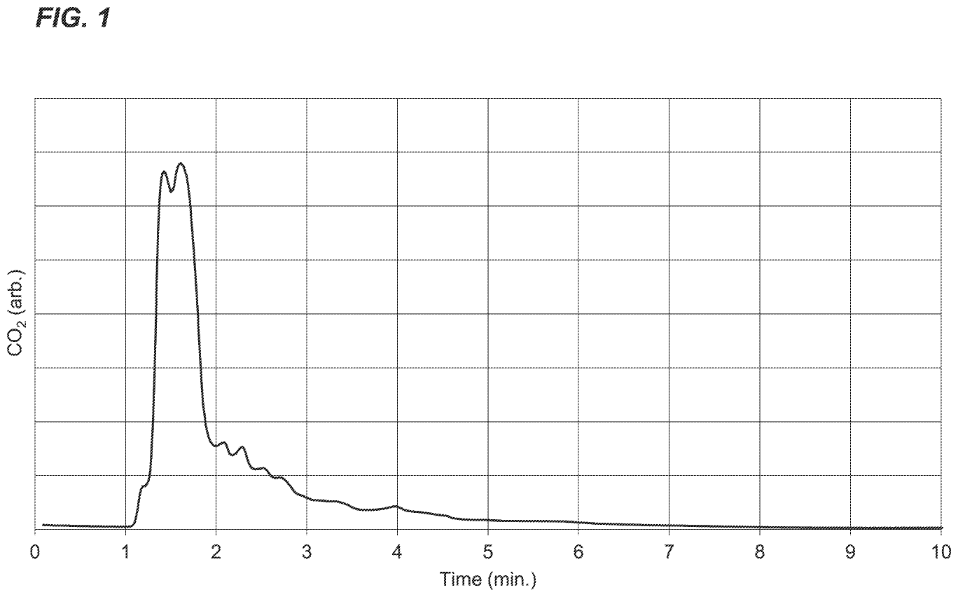

In one embodiment the catalytic reactor for the effluent gases comprises a 3″×¼″ OD stainless steel tube that is filled with a Pt catalyst supported on silica. The reactor is heated to 650° C. and 1-5 sccm of pyrolysis gases are allowed to flow into the reactor. Clean air is supplied at 5 sccm to the reactor. The effluent of the reactor is fed to a NDIR CO₂ detector. During the course of a batch pyrolysis reaction, the measured CO₂ concentration increases to a maximum and then decreases more slowly. The reaction engineer can determine the optimal end point of the reaction from the decay of CO₂ (i.e., total organic carbon off gas), temperature and time. Generally, the minimal level of CO₂ measured that determines the end point of the reaction is less than 100 ppm, or less than 2% of the maximum peak measured values. FIG. 1 depicts, for example, the measurements of CO₂ as a function of time for a reaction run at 400° C. It shows the maximum peak measured and the tailing off of CO₂ being measured. The tailing off of measured CO₂ indicates the ending of the reaction.

The measurement of total organic carbon using this technique is more accurate than other methods such as mass spectrometry or infrared detection because the response factors of all carbon species are the same on a per carbon basis. Whereas mass spectrometry and infrared detectors have responses that depend strongly on the chemical species (e.g., ion and/or absorption cross-section). Mass spectrometry has poor response for low molecular weight species and certain functional groups that impede ionization, especially in the presence of high amounts of water. Infrared detection is also subject to interference from water molecules and it is nearly impossible to deconvolute spectra to quantify the complex mixtures present in pyrolysis gases. Flame ionization detection responds well to many carbon species, but has poor sensitivity to CO, CO₂, formaldehyde and many of the oxygenates present in pyrolysis gases. A Polyarc reactor (oxidation-reduction reactor) could be used in connection with a flame ionization detector to create a universal response to carbon species, but this increases the complexity and cost of implementation, including the requirement for hydrogen supply gas and additional heated zones. Thermal conductivity detection has variable response and it is not possible to calibrate the response without separation of the gases. Gas chromatography can be used to separate gases for individual or grouped quantitation, but this approach requires extensive calibration, adds immense complexity, and prevents real time analysis since discrete samples must be injected with run times typically >5 min. Thus, the total organic carbon technique described herein is the best method for simply quantifying the mixture of organic molecules evolved from pyrolysis reactions.

While the present monitoring process is described in conjunction with a biomass waste conversion reaction, it should be noted that this monitoring method can be applied to any reaction in any reactor where a carbon-based material is pyrolyzed.

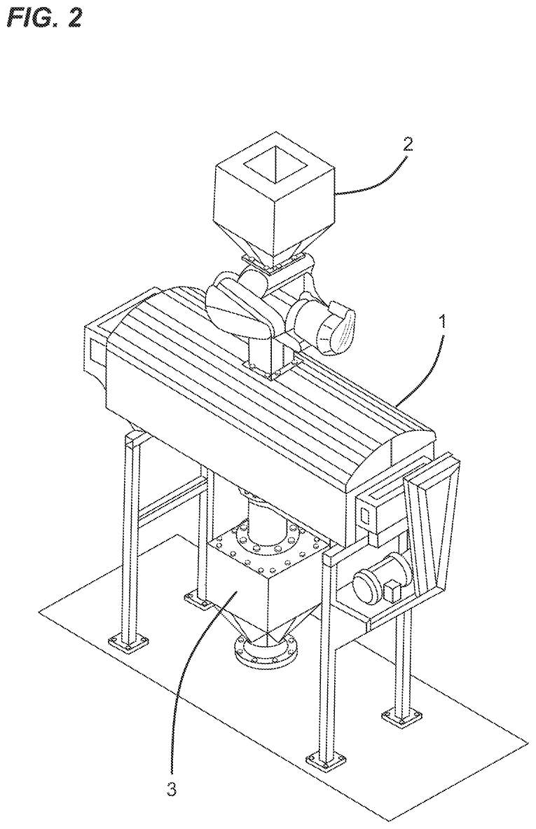

While the present monitoring method can be employed with any suitable reactor and process which affects pyrolysis, or torrefaction, of biomass, a particular example of a process and reactor is provided herein, without limitation. Thus, in one embodiment the process comprises passing the biomass waste into a reactor chamber for pyrolysis. Any suitable pyrolysis reactor can be used. In one embodiment, a preferred reactor for the conversion reaction comprises a twin screw conveyor. The biomass waste is passed along the length of the reactor with heating and mixing. The reactor is heated to a temperature low enough to avoid cracking of the hydrocarbons in the biomass waste, e.g., for 300-450° C., 350-450° C., or more preferably 350-400° C. The twin screw conveyor provides the mixing and conveyance along the length of the reactor. A solid carbon product is then collected from the reactor. The reactor therefore employs a twin screw conveyor to keep the biomass mixed thoroughly while heating, to maintain a uniform temperature profile and faster heat transfer. FIG. 2 depicts one embodiment of a reactor 1. The hopper 2 is for adding biomass, which allows control of the addition. The exit lock hopper 3 allows the solid product to cool prior to handling the product.

The process is semi-batch, meaning a certain amount of material enters the reaction vessel and then reacts/heats for a certain amount of time (10-60 min.), and then exits; subsequently another batch of fresh biomass enters the reaction vessel, and the process is repeated. The mixing action of the screws aids significantly in improving the heat transfer. The reactor is heated from the bottom with one or more burners that combust the gases created during heating of the biomass. This allows the process to be self-heating, without any energy inputs for heating (although electricity is required for the motors to spin the screws). The burner design allows for switching from biomass gases to propane as needed.

The entire vessel can be sealed from the outside air and nitrogen purged. The sealing can be completed 2 with two types of airlocks, a rotary valve, and a butterfly valve, although a rotary valve may be used on both sides 2 and 3 if desired. This can improve nitrogen retention.

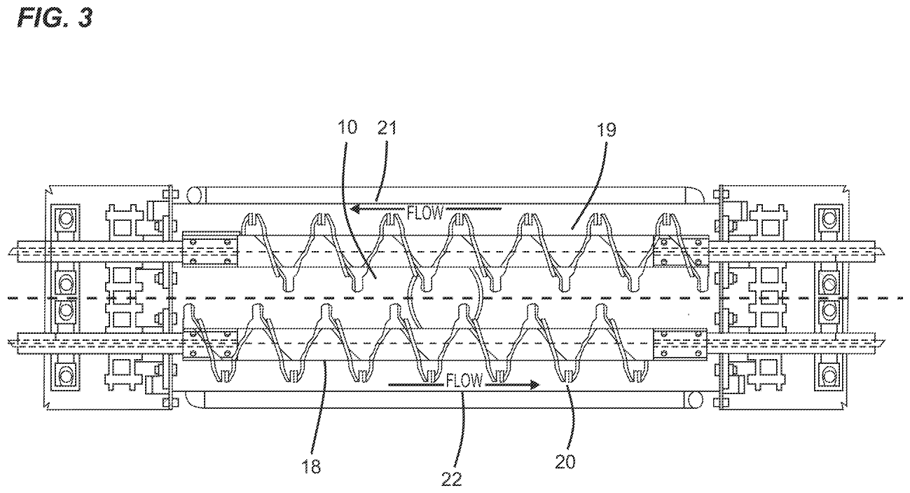

In this embodiment, the reactor is a twin mixing screw conveyor. It is designated primarily for batch-process heating and mixing of biomass. In FIG. 3, the reactor can comprise a twin 7″ ID×4′-0″ long trough 10 that encapsulates two 6″ diameter augers 18 and 19. The augers are designed to convey material in a circular motion (see flow arrows 21 and 22) 360 degrees around the central discharge port as seen in FIG. 3.

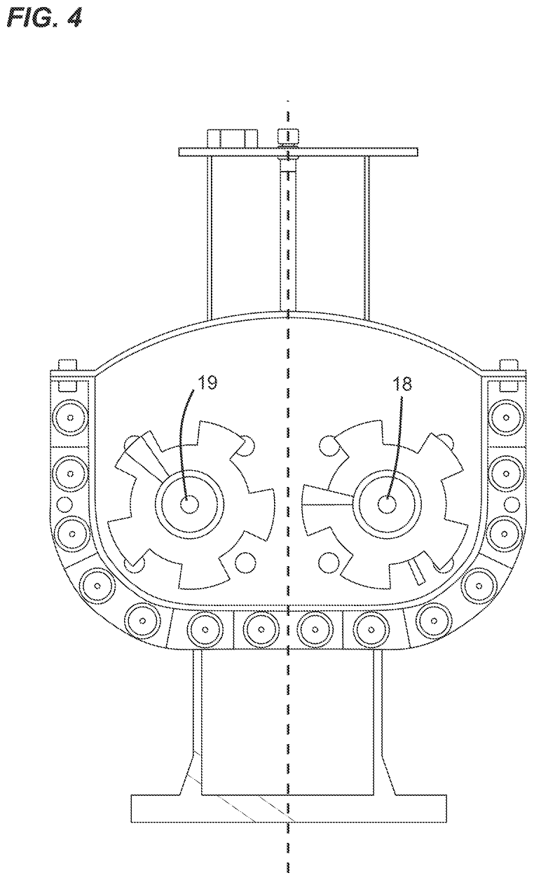

In addition to circulating material in the indicated direction, the cut and folded flights further circulate material, while it conveys, in a 360 degree motion around the central pipe of each auger 18 and 19 (see FIG. 4). The folded parts of the flights act as mixing “fingers” that lift the material as it conveys from 6 o'clock on the flight face to roughly 2 o'clock. This prevents material from conveying en-mass and exposes more material to the bottom of the trough, which promotes heat transfer and quicker reactions times. This enhances greatly the efficiency and effectiveness of the torrefaction of the biomass material.

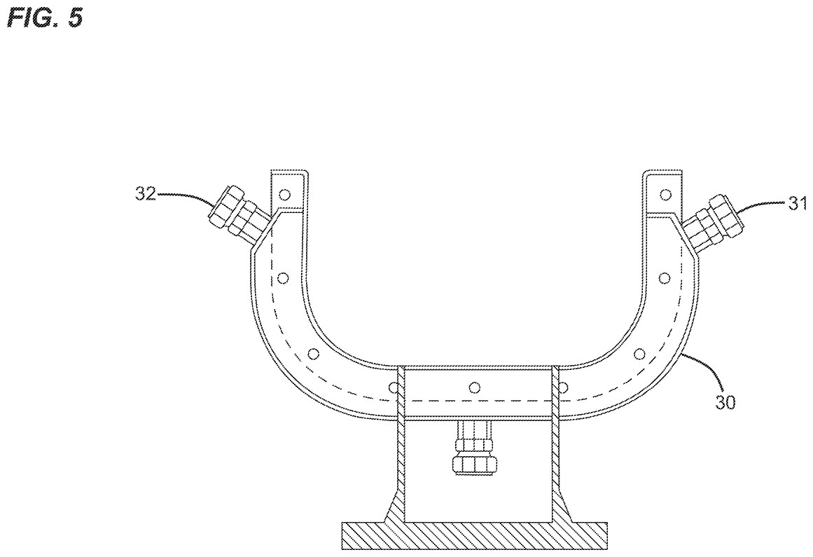

In one embodiment, as shown in FIG. 5, a secondary volume 30 around the reaction vessel is added to include a heat transfer fluid. This fluid provides two functions, first to improve the heat transfer by allowing convective flows of fluid around the outside of the reactor, and second to provide an energy storage medium that will buffer the transients in heat provided by the burner as the combustion gases from the reaction change over time during the reaction. In one embodiment, a blend of 60% NaNO₃ and 40% KNO₃ (by mass), commonly referred to as solar salt is used as the heat transfer fluid. This molten salt has the advantage of lower corrosiveness (compared with chlorides), its relatively low melting point of 220° C., and its decent heat storage ability (heat capacity). In effect, the system has a thermal battery around the reactor that limits temperature swings. FIG. 5 depicts the containment around the reactor for a heat transfer fluid, such as a molten salt heat transfer fluid. Addition of heat transfer fluid can be made at parts 31 and 32.

In another embodiment, the fluid can be pumped around the reactor and through the screws to increase heat transfer. The twin screws can be designed for this.

In another embodiment, the heat capacity of the entire system is improved by using a solid metal rod for the screws and relatively thick stainless steel on the body. The entire reactor is then insulated from the atmosphere with thick mineral wool. Thermal breaks are provided between the reactor and valves in the form of a long cylinder that allows material to pass through, but are thin walled to limit heat transfer (some portion of these are insulated). This also buffers transients. The reactor operates between 300-450° C., but ideally 350-400° C. This reactor provides improved heat transfer, reliability, efficiency and economics.

Once the reaction is stopped based upon the monitoring of the reaction, the carbon product is then collected from the reactor. It can be spread as solid carbon on open ground if desired, but this may not avoid carbon in the atmosphere as CO₂ for as long as needed. In one embodiment, the solid carbon product recovered from the reactor is collected and then shipped to a site where it can be buried and covered. The site can be an old mine or quarry. The site underground can also be created, e.g., dug, in an appropriate area and at an appropriate depth. Such sites can increase permanence of the carbon from 100 to 1 million years, or more. The overall process allows a most efficient and economic means of removing carbon, and CO₂, from the atmosphere. The present monitoring method also provides an accurate surrogate for reaction progress. More accurate reaction monitoring allows for improved reaction control, optimization and the ability to readily utilize variable feedstocks.

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise that as specifically described without actually departing from the spirit and scope of the present invention as defined by the appended claims.

What is claimed is:

1. A process for measuring the progress of a pyrolysis reaction in a reactor by catalytic oxidation of reaction products and subsequent measurement of the resulting CO₂.

2. The process of claim 1, wherein the progress of the reaction is monitored by passing organic molecules in the effluent of the reactor to a catalytic reactor for catalytic combustion of said organic molecules in the effluent to CO₂.

3. The process of claim 2, wherein adjustments to the reaction are made based on the monitoring of the reaction.

4. The process of claim 3, wherein the monitoring permits an adjustment to the length of the reaction.

5. The process of claim 2, wherein the reaction is shut down once a minimal level of CO₂ in the effluent of the reactor is measured.

6. The process of claim 1, wherein the reactor has a secondary volume of heat transfer fluid around the reactor.

7. The process of claim 6, wherein the heat transfer fluid comprises a solar salt composition.

8. The process of claim 1, wherein the reactor comprises a twin screw conveyor which comprises twin screws with cuts and folds.

9. The process of claim 8, wherein heat transfer fluid is passed internally through the twin screws of the twin screw conveyor.

10. The process of claim 1, wherein a solid carbon product is collected from the reactor and is buried underground and covered.

11. The process of claim 2, wherein the catalytic reactor contains a combustion catalyst.

12. The process of claim 11, wherein the combustion catalyst comprises Pt, Pd, Rh and/or Co.

13. The process of claim 11, wherein the catalytic oxidation is conducted in a reactor heated to 450-650° C.

14. A reactor for converting biomass to a solid carbon product comprising:
a reactor chamber that can be heated to pyrolysis temperatures;
a twin screw conveyer in the reactor chamber;
a catalytic reactor in connection with the reactor chamber such that the catalytic reactor receives effluent from the reactor;
a CO₂ detector in connection with the catalytic reactor such that the CO₂ detector receives effluent from the catalytic reactor.

15. The reactor of claim 14, wherein the catalytic reactor comprises a combustion catalyst selected from Pt, Pd, Rh and/or Co.
